(19) United States
(12) Patent Application Publication
(10) Pub. No.: US 2022/0399104 A1
(43) Pub. Date: Dec. 15, 2022

(54) DRIVE-THROUGH TREATMENT SYSTEM

(71) Applicants: Hyundai Motor Company, Seoul (KR); Kia Corporation, Seoul (KR)

(72) Inventor: Joon Young Kim, Seongnam (KR)

(21) Appl. No.: 17/545,281

(22) Filed: Dec. 8, 2021

(30) Foreign Application Priority Data
Jun. 11, 2021 (KR) 10-2021-0076047

(51) Int. Cl. G16H 40/20; G16H 40/67; G07C 11/00; G06V 20/54; G06V 20/62

(52) U.S. Cl. CPC G16H 40/20; G16H 40/67; G07C 11/00; G06V 20/54; G06V 20/625; G07C 2011/04; G06V 2201/08

(57) ABSTRACT

A drive-through treatment system for a drive-through clinic includes: a plurality of monitoring devices positioned at an inlet of a plurality of treatment lines; a plurality of treatment devices positioned on a plurality of medical shelters in each of the plurality of treatment lines; a plurality of outlet devices positioned at an outlet of the plurality of treatment lines; and a treatment line determining device for determining a treatment line into which a target vehicle to enter the drive-through clinic will enter among the plurality of treatment lines based on information received from a plurality of monitoring devices and a plurality of outlet devices. Each of the plurality of monitoring devices may check a treatment start, a treatment progress situation, and a treatment speed of vehicles that have entered the treatment line from the inlet of the corresponding treatment line to be transmitted to the treatment line determining device.

### CROSS-REFERENCE TO RELATED APPLICATION

This application claims under 35 U.S.C. § 119 the benefit of Korean Patent Application No. 10-2021-0076047 filed in the Korean Intellectual Property Office on Jun. 11, 2021, the entire contents of which are incorporated herein by reference.

### BACKGROUND

### (a) Technical Field

The present disclosure relates to a drive-through treatment system.

### (b) Description of the Related Art

In the case of a drive-through examination/treatment service, it is necessary to move vehicles quickly according to an examination/treatment process. Typically, a plurality of lines for the examination/treatment are built in parallel. However, vehicle movement may not be smooth due to a delay of the examination/treatment in a certain line among the plurality of examination/treatment lines. For example, when a new vehicle is moved into a corresponding line, a problem of aggravating a bottleneck phenomenon may occur.

The above information disclosed in this Background section is only for enhancement of understanding of the background of the disclosure, and therefore it may contain information that does not form the prior art that is already known in this country to a person of ordinary skill in the art.

### SUMMARY

A drive-through treatment system is configured to optimize a time required for examination/treatment.

A drive-through treatment system for a drive-through clinic according to one aspect of the disclosure includes a plurality of monitoring devices positioned at an inlet of a plurality of treatment lines; a plurality of treatment devices positioned on a plurality of medical shelters in each of a plurality of treatment lines; a plurality of outlet devices positioned at an outlet of a plurality of treatment lines; and a treatment line determining device for determining a treatment line into which a target vehicle to enter the drive-through clinic will enter among the plurality of treatment lines based on the information received from the plurality of monitoring devices and the plurality of outlet devices. Each of the plurality of monitoring devices may check a treatment start, a treatment progress situation, and a treatment speed of vehicles that have entered the treatment line from the inlet of the corresponding treatment line to be transmitted to the treatment line determining device, and each of the plurality of outlet devices may check a final treatment time of the vehicles exiting the treatment line from the outlet of the corresponding treatment line, a remaining time moving from the inlet to the outlet, and a treatment result to be transmitted to the treatment line determining device.

Each of the plurality of monitoring devices may receive queue status information, a treatment required time for each of the vehicles in the queue, and treatment information of a doctor from each of the plurality of treatment devices of the corresponding treatment line, and synthesize the received information to be transmitted to the treatment line determining device. The queue may be a list of the vehicles that have entered the clinic where each of the plurality of treatment devices of the corresponding treatment line are positioned, the queue status information includes information on the number of the vehicles being treated in the corresponding medical shelter and a time of entering and exiting the queue of each of the vehicles, and the treatment required time for each of the vehicles in the queue may be a treatment required time for each of the vehicles of the corresponding medical shelter.

Each of the plurality of monitoring devices may analyze and check a plurality of treatment information sets received from the plurality of treatment devices of the corresponding treatment line, and may include a data collection unit classifying and collecting the identified treatment information according to a predetermined collection format.

The data collection unit may acquire information on the treatment status and the treatment required time of the medical shelter in which each of the plurality of treatment devices of the corresponding treatment line is positioned based on a plurality of queue situation information sets of the corresponding treatment line and the treatment required time for each of the vehicles in a plurality of queues.

The data collection unit may collect the number of the vehicles receiving treatment from each medical shelter according to each of the plurality of queue situation information sets and the treatment required time for each of the vehicles in the plurality of queues to obtain a total treatment required time of each of the plurality of medical shelters of the corresponding treatment line.

Each of the plurality of monitoring devices may further include a data formatter for receiving information from the data collection unit and converting the received information into a specific data format.

Each of the plurality of monitoring devices may further include, when a patient with a serious problem enters the corresponding treatment line, or there is treatment information instructing an occurrence of an emergency situation during the treatment, among the plurality of treatment information sets received from the plurality of treatment devices, an emergency processing unit to inform this to the outside, and each data structure of the plurality of treatment information sets may include a data field capable of recognizing an emergency situation by the emergency processing unit.

Each of the plurality of outlet devices may classify the patient as an emergency patient based on the information received from the corresponding monitoring device among the plurality of monitoring devices, and may guide the patient to go to an emergency room or a higher-level clinic.

Each of the plurality of outlet devices may include: an entry detection unit that checks the type and the number plate of the target vehicle entering the outlet of the corresponding treatment line, and the patient, and requests and receives the time-related information and the treatment information for the identified patient from the corresponding monitoring device; and a parsing unit for separating the time-related information and the treatment information. The time-related information may include the information about the queue entry time point, the queue exiting time point, and a current queue value of the target vehicle in each of the plurality of medical shelters of the corresponding treatment line.

Each of the plurality of outlet devices may further include: a patient state analyzing unit that determines the state of the patient who has entered the outlet, and determines the final examination result in consideration of the identified patient state along with the treatment information received from the parsing unit; and a queue delay analyzing unit that collects the time-related information to check the required time for each queue of the plurality of medical shelters of the corresponding treatment line and analyzes whether there is a delay in receiving the treatment for the target vehicle.

The queue delay analyzing unit may determine that there is a delay in the treatment at the medical shelter corresponding to the corresponding queue if the time from the time point when the target vehicle enters the queue corresponding to each of the plurality of medical shelters of the corresponding treatment line to the time point when the target vehicle comes out of the corresponding queue is greater than or equal to a predetermined threshold value.

The queue delay analyzing unit may determine that there is a delay in moving between two adjacent medical shelters corresponding to the previous queue and the corresponding queue when the time difference between the time when the target vehicle exits from the previous queue and the time when the target vehicle enters the corresponding queue is greater than or equal to a predetermined threshold value based on the queue corresponding to each of the plurality of medical shelters of the corresponding treatment line.

The treatment line determining device may include: a data reallocation unit performing data processing and data formatting on the data received from the target vehicle; a patient status determining unit for receiving the patient status of the target vehicle and position information on the movement path of the target vehicle from the data reallocation unit, and determining a status level indicating the patient's status level of the target vehicle; and a queue analyzing unit for analyzing the current delay situation in the plurality of medical shelters in each of the plurality of treatment lines. The queue may be a list of the vehicles that have entered the clinic where each of the plurality of treatment devices of each of the plurality of treatment lines is positioned.

The queue analyzing unit may receive each queue status information set of the entire medical shelter that is the plurality of medical shelters of an entirety of the plurality of treatment lines from the plurality of monitoring devices, and derive the number of each queue of the entire medical shelter, the waiting times, and the treatment time.

The queue analyzing unit, in two adjacent medical shelters of the plurality of medical shelters in one treatment line, may derive a period between the time point when the target vehicle exits from the previous queue, which is the queue of the medical shelter positioned first in the target vehicle progress direction and the time point when the target vehicle enters the next queue, which is the queue of the medical shelters positioned next in the target vehicle progress direction, as the waiting time of the next medical shelter.

The queue analyzing unit may derive the period between the time point when the target vehicle enters the queue of the medical shelter and the time point when it leaves the same queue as the treatment time of the medical shelter.

The treatment line determining device may further include a delay analyzing unit for determining a delay level for each of the plurality of treatment lines based on the number of the queues for each of the entire medical shelter, the waiting time, and the treatment time, respectively.

The delay analyzing unit may derive the number of the queues by adding up each number of the entire medical shelter that is the plurality of medical shelters in an entirety of the plurality of treatment lines, and derive an average of the waiting time and the treatment time of the plurality of medical shelters constituting each of the plurality of treatment lines.

The delay analyzing unit may compare each of the queue number, the average waiting time, and the average treatment time of each of the plurality of treatment lines with a corresponding reference value to determine a delay level indicating a delay degree of each of the plurality of treatment lines based on each of the queue number, the average waiting time, and the average treatment time.

The delay analyzing unit may determine that the treatment status of the treatment line is busy when the number of the queues in one of the plurality of treatment lines is greater than the first reference value, and the treatment status of the treatment line is normal when it is less than the first reference value.

The delay analyzing unit may determine that the treatment status of the treatment line is normal when the average waiting time of one of the plurality of treatment lines is less than or equal to the second reference value, the treatment status of the treatment line is delayed when the average waiting time is greater than the second reference value, and the treatment status of the treatment line is judged as serious when the average waiting time is greater than the third reference value R3, which is greater than the second reference value.

The delay analyzing unit may determine that the treatment status is delayed if the average treatment time of one of the plurality of treatment lines is greater than the fourth reference value, and the treatment status is normal if the average treatment time is less than the fourth reference value.

The treatment line determining device may further include a patient analyzing unit determining the patient status level by collecting the final examination result for the patient status received from the plurality of outlet devices and the treatment information received from the plurality of monitoring devices, derives the total treatment time that is the sum of the treatment times of the plurality of medical shelters constituting the treatment line in which the treatment of the patient has been performed and the average treatment time that is the average thereof, and indexes the average treatment time and the total treatment time to the patient status level.

The treatment line determining device may further include a line determining unit to determine the treatment line of the target vehicle among the plurality of treatment lines based on the delay level of each of the plurality of treatment lines received from the delay analyzing unit and the status level received from the patient status determining unit.

The line determining unit may calculate a weighted delay level by multiplying the delay level for each of the plurality of treatment lines by a weight value corresponding to the treatment line, and determine the treatment line having the smallest value among the weighted delay levels of the plurality of treatment lines as the treatment line of the target vehicle.

The line determining unit may set the weight value of each of the plurality of treatment lines as a state level average for the patients of n vehicles which have recently entered each of the plurality of treatment lines.

The treatment line determining device may further include: a patient analyzing unit determining the patient status level by collecting the final examination result for the patient status received from the plurality of outlet devices and the treatment information received from the plurality of monitoring devices, derives the total treatment time that is the sum of the treatment times of the plurality of medical shelters constituting the treatment line in which the treatment of the patient has been performed and the average treatment time that is the average thereof, and indexes the average treatment time and the total treatment time to the patient status level; and a line determining unit for determining the treatment line of the target vehicle among the plurality of treatment lines based on the delay level of each of the plurality of treatment lines received from the delay analyzing unit, the status level received from the patient status determining unit, and the indexed patient status level received from the patient analyzing unit.

Through various embodiments according to the present disclosure, it is possible to provide the drive-through treatment system that may optimize the time required for the examination/treatment.

### BRIEF DESCRIPTION OF THE DRAWINGS

FIG. 1 is a view showing a drive-through treatment system according to an embodiment.

FIG. 2 is a view showing a monitoring device according to an embodiment.

FIG. 3 is a view showing an outlet device according to an embodiment.

FIG. 4 is a view showing a treatment line determining device according to an embodiment of the present disclosure.

FIG. 5 is a flowchart showing an operation of the delay analyzing unit according to an embodiment.

### DETAILED DESCRIPTION

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g., fuels derived from resources other than petroleum). As referred to herein, a hybrid vehicle is a vehicle that has two or more sources of power, for example, both gasoline-powered and electric-powered vehicles.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements. In addition, the terms “unit”, “-er”, “-or”, and “module” described in the specification mean units for processing at least one function and operation, and can be implemented by hardware components or software components and combinations thereof.

Further, the control logic of the present disclosure may be embodied as non-transitory computer readable media on a computer readable medium containing executable program instructions executed by a processor, controller or the like. Examples of computer readable media include, but are not limited to, ROM, RAM, compact disc (CD)-ROMs, magnetic tapes, floppy disks, flash drives, smart cards and optical data storage devices. The computer readable medium can also be distributed in network coupled computer systems so that the computer readable media is stored and executed in a distributed fashion, e.g., by a telematics server or a Controller Area Network (CAN).

The present disclosure relates to a drive-through treatment system for selecting an optimal treatment line among the plurality of treatment lines for a newly entering vehicle when the plurality of lines (hereinafter, treatment lines) for a drive-through examination/treatment service are built in parallel. By selecting the optimal treatment line, a treatment time may be optimized. The present disclosure may determine a status of each treatment line by collecting information obtained from each of an inlet and an outlet of each treatment line. If an unusual occurrence occurs in any one of the plurality of treatment lines, it is possible to prevent a bottleneck phenomenon in the treatment line by taking appropriate measures.

The present disclosure will be described more fully hereinafter with reference to the accompanying drawings, in which embodiments of the disclosure are shown. As those skilled in the art would realize, the described embodiments may be modified in various different ways, all without departing from the spirit or scope of the present disclosure.

Hereinafter, embodiments disclosed in the present specification will be described in detail with reference to the accompanying drawings. In the present specification, the same or similar components will be denoted by the same or similar reference numerals, and an overlapped description thereof will be omitted. Further, in describing embodiments of the present specification, when it is determined that a detailed description of the well-known art associated with the present disclosure may obscure the gist of the present disclosure, it will be omitted. In addition, the accompanying drawings are provided only in order to allow embodiments disclosed in the present specification to be easily understood and are not to be interpreted as limiting the spirit disclosed in the present specification, and it is to be understood that the present disclosure includes all modifications, equivalents, and substitutions without departing from the scope and spirit of the present disclosure.

Terms including ordinal numbers such as first, second, and the like will be used only to describe various components, and are not to be interpreted as limiting these components. The terms are only used to differentiate one component from other components.

It is to be understood that when one component is referred to as being “connected” or “coupled” to another component, it may be connected or coupled directly to another component or be connected or coupled to another component with the other component intervening therebetween.

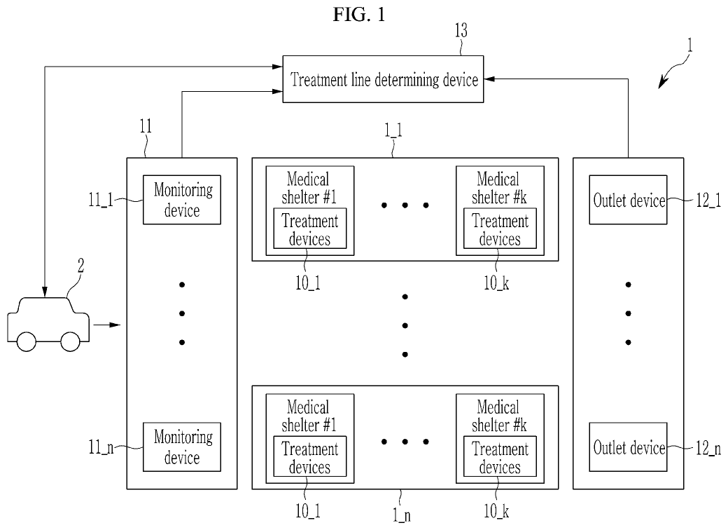

FIG. 1 is a view showing a drive-through treatment system according to an embodiment.

A plurality of treatment lines 1_1 to 1_n (where n is a natural number of 2 or more) is positioned in a drive-through clinic to which a drive-through treatment system 1 is applied. A monitoring device 11 is positioned at an inlet of each of the plurality of treatment lines 1_1 to 1_n, next a plurality of medical shelters #1 to #k (k is a natural number of 2 or more) is positioned, and an outlet device 12 is positioned at an outlet of each of the plurality of treatment lines 1_1 to 1_n. A patient in the vehicle may receive a treatment at the plurality of medical shelters #1 to #k between the inlet and the outlet while moving from the inlet to the outlet of each treatment line with the vehicle.

In each of the plurality of medical shelters #1 to #k, an event (storage, a reservation, etc.) related to any one of an inspection, a treatment, or an inspection and treatment is performed, and appropriate devices are provided according to work performed by each medical shelter. Hereinafter, each piece of equipment provided in each medical shelter is defined as a treatment device. When the medical treatment is performed in the medical shelter, a doctor may be positioned in the medical shelter, and when an examination is conducted in the medical shelter, a doctor or an engineer specializing in the examination may be positioned in the medical shelter.

As shown in FIG. 1, the drive-through treatment system 1 includes a plurality of monitoring devices 11_1 to 11_n positioned at the inlet of the plurality of treatment lines 1_1 to 1_n, a plurality of treatment devices 10_1 to 10_k positioned at the plurality of medical shelters #1 to #k in each of the plurality of treatment lines 1_1 to 1_n, a plurality of outlet devices 12_1 to 12_n positioned at the outlet of the plurality of treatment lines 1_1 to 1_n, and a treatment line determining device 13.

Each of the plurality of monitoring devices 11_1 to 11_n may check a treatment start, a treatment progress, and a treatment speed of the vehicles that have entered the treatment line in the inlet of the corresponding treatment line, and may transmit the check to the treatment line determining device 13.

Each of the plurality of outlet devices 12_1 to 12_n may check the final treatment time of the vehicles passing through the treatment line from the outlet of the corresponding treatment line, the remaining time that the vehicles move from the inlet to the outlet, and the treatment result to be transmitted to the treatment line determining device 13.

The treatment line determining device 13 may determine the treatment line into which the corresponding vehicle 2 enters to receive the treatment from the plurality of treatment lines based on information received from the plurality of monitoring devices 11_1 to 11_n and the plurality of outlet devices 12_1 to 12_n. The vehicle may transmit information about the patient's condition (body temperature, blood pressure, current symptoms, etc.) and personal information for identifying the patient to the treatment line determining device 13 through a wireless communication. Some of the patient's status information and personal information may be transmitted from the patient's personal portable terminal to the treatment line determining device 13.

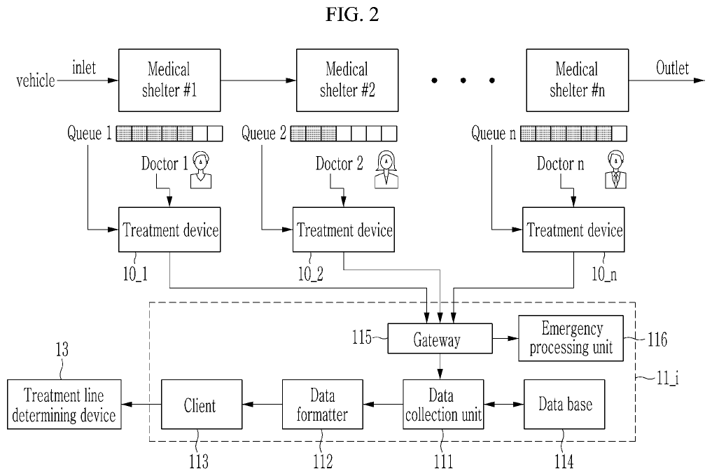

FIG. 2 is a view showing a monitoring device according to an embodiment.

In FIG. 2, one of the plurality of monitoring devices 11_1 to 11_n (11_i, i is one of natural numbers from 1 to n) is shown. Since the plurality of monitoring devices 11_1 to 11_n have the same configuration, only one monitoring device is described.

As shown in FIG. 2, the monitoring device 11_i may receive queue status information, a treatment time required for each vehicle in the queue, and the doctor's treatment information from each of the plurality of treatment devices 10_1 to 10_k of the i-th treatment line, and collect the received information to be transmitted to the treatment line determining device 13. The monitoring device 11_i may include a management layer for collecting the information received from each of the plurality of treatment devices 10_1 to 10_k. The queue is a list of vehicles that have entered the clinic where each of the plurality of treatment devices 10_1 to 10_k is positioned. The queue status information may include information on the number of vehicles being treated in the corresponding medical shelter, and the timing of entering and exiting the queue of each vehicle. The treatment required time for each vehicle in the queue may mean the treatment required time for each vehicle of the corresponding medical shelter. The treatment required time includes a waiting time to receive the treatment and a treatment time for receiving the treatment.

The monitoring device 11_i includes a data collection unit 111, a data formatter 112, a client 113, a database 114, a gateway 115, and an emergency processing unit 116.

A plurality of queue status information sets, the treatment required time for each vehicle in a plurality of queues, and a plurality of treatment information sets are transmitted from the plurality of treatment devices 10_1 to 10_k through the gateway 115 to the data collection unit 111.

The data collection unit 111 analyzes and grasps the plurality of treatment information sets received from the plurality of treatment devices 10_1 to 10_k, and sorts and collects the identified treatment information according to a predetermined collection format. The plurality of treatment information sets received from the plurality of treatment devices 10_1 to 10_k may be generated with a level that is standardized. For example, information about the age and weight of the patient, the patient's current symptoms (body temperature, blood pressure, etc.) and information (a severity of high/medium/low or none) about the patient's underlying disease (diabetes, high blood pressure, heart failure, etc.) may be included. The data collection unit 111 may acquire the information for the treatment situation and the treatment required time of the medical shelter in which each of the plurality of treatment devices 10_1 to 10_k is positioned based on a plurality of queue situation information sets and the treatment required time for each vehicle in the plurality of queues. For example, the data collection unit 111 may know the number of the vehicles treated at each medical shelter according to the queue situation information, and may know the total treatment required time of each medical shelter by collecting the treatment required time for each vehicle in the queue.

The data collection unit 111 may store the collected data in the database 114. In addition, the database 1140 may store the information for each of the plurality of treatment devices 10_1 to 10_k and the plurality of treatment information sets received from the plurality of treatment devices 10_1 to 10_k.

The data formatter 112 receives the information collected from the data collection unit 111 and converts the received information into a specific data format (e.g., a JSON format). The specific data type may be set as one of data types that may be processed by the treatment line determining device 13.

The client 113 transmits the data output from the data formatter 112 to the treatment line determining device 13.

The emergency processing unit 116 may receive the plurality of treatment information sets through the gateway 115, and when there is treatment information indicating that a serious patient enters or an emergency situation occurs during the treatment, notify it to the outside. In this case, the outside may be an outside of the drive-through clinic as well as an outside of the treatment line. Then, the doctor suitable for the emergency situation may be urgently placed in the medical shelter. Each data structure of the plurality of treatment information sets may include a predetermined data field so that the emergency processing unit 116 may recognize an emergency situation.

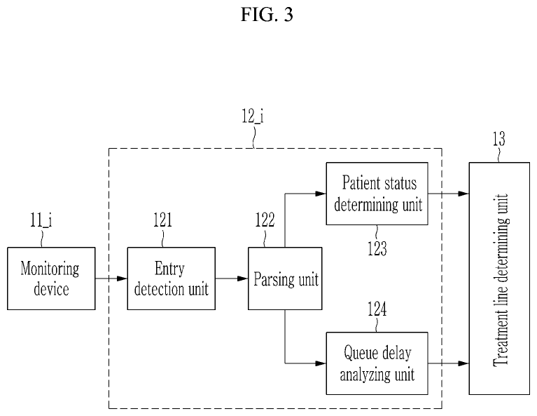

FIG. 3 is a view showing an outlet device according to an embodiment.

FIG. 3 shows the outlet device 12_i positioned at the outlet of the i-th treatment line among the plurality of outlet devices 12_1 to 12_n.

The outlet device 12_i determines follow-up actions for the patients who have been examined and treated in the plurality of medical shelters #1 to #k. For example, based on the information received from the monitoring device 11_i, the patient may be classified as an emergency patient and guided to go to the emergency room, or may be guided to go to a higher clinic but not as an emergency.

The outlet device 12_i includes an entry detection unit 121, a parsing unit 122, a patient state analyzing unit 123, and a queue delay analyzing unit 124.

The entry detection unit 121 checks a type and a number plate of the vehicle entering the outlet of the treatment line, and the patient, requests the time-related information and the treatment information for the patient, and receives the time-related information and the treatment information, which are confirmed from the monitoring device 11_i. The entry detection unit 121 transfers the received information to the parsing unit 122. The time-related information may include information about a queue entry time point, a queue exiting time point, and a current queue value of the vehicle in each of the plurality of medical shelters #1 to #k.

The parsing unit 122 separates the time-related information and the treatment information, and transmits the time-related information to the queue delay analyzing unit 124 and the treatment information to the patient state analyzing unit 123.

The patient state analyzing unit 123 determines the state of the patient who has entered the outlet, and determines the final examination result in consideration of the identified patient state together with the treatment information received from the parsing unit 122. The state of the patient may be a body temperature of the patient. The patient state analyzing unit 123 may derive the body temperature of the patient by analyzing thermal imaging acquired by the thermal imaging camera provided at the outlet. The patient state analyzing unit 123 may classify an emergency patient or a patient who needs to go to a higher-level clinic based on the treatment information and the state of the patient.

The queue delay analyzing unit 124 may collect the time-related information, check the required time for each queue of the plurality of medical shelters #1 to #k, and analyze whether there is a delay in receiving treatment for the corresponding vehicle. For example, the queue delay analyzing unit 124 may know the time the corresponding vehicle enters and exits each of the plurality of medical shelters #1 to #k with the time that it enters and exits the queue in each of the plurality of medical shelters #1 to #k. The queue delay analyzing unit 124 may determine that there is a delay in the treatment at the medical shelter corresponding to the specific queue if the time from the entry time point into the specific queue for one of the plurality of medical shelters #1 to #k and the exit time from the specific queue is greater than or equal to a predetermined threshold value. The queue delay analyzing unit 124 may determine that there is a delay in the moving between two adjacent medical shelters corresponding to the previous queue and the specific queue when there is a difference between the exiting time point from the previous queue to the entry time point into the specific queue based on the specific queue. The queue delay analyzing unit 124 may analyze the occurrence reason for the delay by acquiring the information necessary for the analysis from the outside. The queue delay analyzing unit 124 may transmit the queue status information of each of the plurality of medical shelters #1 to #k to the treatment line determining device 13. The queue status information may include the information about the time point when the vehicle enters the queue, the time point when the vehicle leaves the queue, and the number of vehicles remaining in the queue.

The information about the patient state, which is the output of the patient state analyzing unit 123, and the information on whether the delay occurs and the reason, which are the output of the queue delay analyzing unit 124, are transmitted to the treatment line determining device 13.

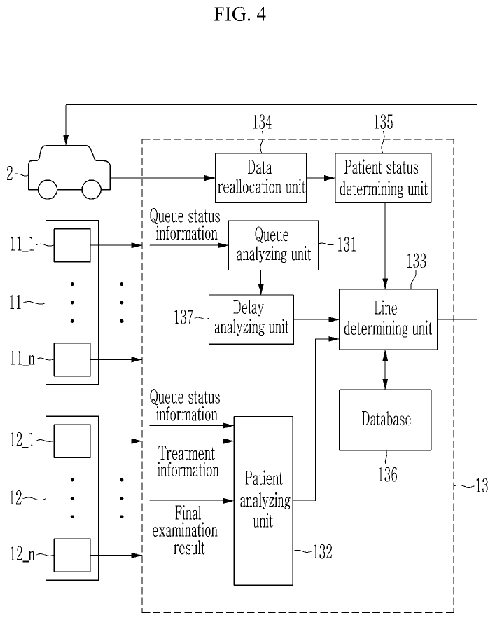

FIG. 4 is a view showing a treatment line determining device according to an embodiment of the present disclosure.

The treatment line determining device 13 includes a queue analyzing unit 131, a patient analyzing unit 132, a line determining unit 133, a data reallocation unit 134, a patient status determining unit 135, a database 136, and a delay analyzing unit 137.

The data reallocation unit 134 performs data processing and data formatting for the data received from the target vehicle 2. The data received from the vehicle is in a form of binary or text. This may not be a data format suitable for the treatment line determining device 13. The data reallocation unit 134 converts the data received from the vehicle into a data format suitable for the patient status determining unit 135. For example, the data input to the data reallocation unit 134 may be in the form of binary or text, and the data output from the data reallocation unit 134 may be of the JOSN type. The data input from the vehicle includes the information about the state (age, weight, body temperature, blood pressure, facial condition, an underlying disease, etc.) of the patient, position information about the path in which the vehicle has moved, and basic medical information about the patient.

The patient status determining unit 135 receives the patient status on the target vehicle 2 and the position information on the path in which the target vehicle 2 has moved from the data reallocation unit 134, and determines whether the patient status is at a certain level (e.g., Level 1, 2, 3, 4, and an emergency). The patient status determining unit 135 transmits a status level indicating the patient status to the line determining unit 133.

The queue analyzing unit 131 may analyze a current delay situation in the plurality of medical shelters #1 to #k of each of the plurality of treatment lines. The queue analyzing unit 131 receives the queue situation information of each of the plurality of medical shelters #1 to #k (hereinafter, the entire medical shelter) of the plurality of treatment lines from the plurality of monitoring devices 11_1 to 11_n, and may derive the number of the queues of the entire medical shelter, the waiting time, and the treatment time. The queue analyzing unit 131, in two adjacent medical shelters among the plurality of medical shelters in each treatment line, may derive the period between the time that the vehicle leaves the queue (the previous queue) of the medical shelter positioned first in the vehicle progress direction and the time that the corresponding vehicle enters the queue (the next queue) of the medical shelter positioned next in the vehicle progress direction as the waiting time of the next medical shelter. The queue analyzing unit 131 may derive the period between the time when the vehicle enters the queue of the medical shelter and the time when the vehicle leaves the same queue as the treatment time of the medical shelter. In this way, the waiting time and the treatment time for the entire medical shelter may be derived. The queue analyzing unit 131 may convert the information on the number of the queues, the waiting time, and the medical hours for the entire medical shelter into a specific data format (e.g., the JSON format) to be transmitted to the delay analyzing unit 137. In addition, the queue analyzing unit 131 may transmit and store the corresponding information to the database 136.

The delay analyzing unit 137 may determine a delay level for each of the plurality of treatment lines based on the number of the queues, the waiting time, and the treatment time for each of the entire medical shelters, respectively. The delay analyzing unit 137 derives the number of the queues by adding all the numbers of each of the entire medical shelters, and derives the average of the waiting time and the treatment time of the plurality of medical shelters constituting each of the plurality of treatment lines. The delay analyzing unit 137 may compare each of the queue number, the average waiting time, and the average treatment time with corresponding reference values to determine the treatment situation based on each of the queue number, the average waiting time, and the average treatment time. The delay analyzing unit 137 may determine a delay level indicating the delay degree of each of the plurality of treatment lines by synthesizing the treatment situation based on each of the queue number, the average waiting time, and the average treatment time for each of the plurality of treatment lines.

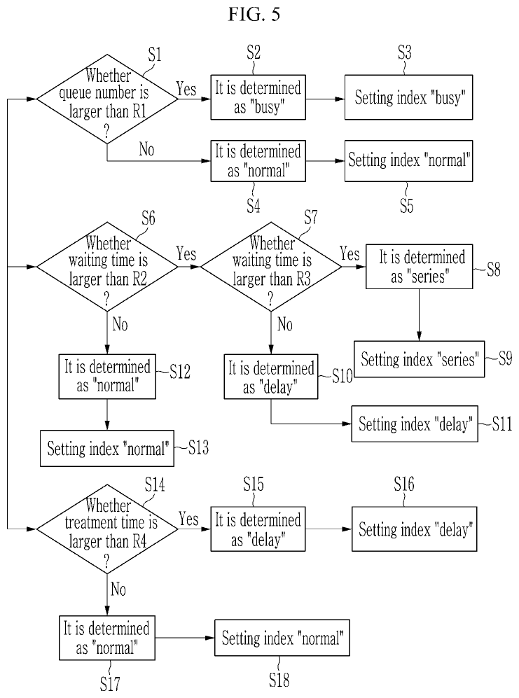

FIG. 5 is a flowchart showing an operation of a delay analyzing unit according to an embodiment.

The delay analyzing unit 137 determines whether the number of the queues corresponding to one of the plurality of treatment lines (hereinafter, referred to as the corresponding treatment line) is greater than a first reference value (S1).

As the determination result of the step S1, if the number of the queues is greater than the first reference value R1, the delay analyzing unit 137 determines the treatment status of the treatment line as ‘busy’ (S2). The delay analyzing unit 137 may set the index to ‘busy’ for the number of the queues in the treatment line (S3).

As the determination result of the step S1, if the number of the queues is less than or equal to the first reference value R1, the delay analyzing unit 137 determines the treatment status of the corresponding treatment line as ‘normal’ (S4). The delay analyzing unit 137 may set the index to ‘normal’ for the number of the queues in the treatment line (S5).

The delay analyzing unit 137 determines whether the average waiting time corresponding to the corresponding treatment line is greater than the second reference value R2 (S6).

As the result of the determination in the step S6, if the average waiting time is greater than the second reference value R2, the delay analyzing unit 137 determines whether the average waiting time is greater than the third reference value R3 (S7).

As the judgment result of the step (S7), if the average waiting time is greater than the third reference value R3, the delay analyzing unit 137 determines the treatment status of the treatment line as ‘serious’ (S8). The delay analyzing unit 137 may set the index to ‘severe’ for the average waiting time of the treatment line S9.

As the result of the determination in the step (S7), if the average waiting time is less than or equal to the third reference value R3, the delay analyzing unit 137 determines the treatment status of the corresponding treatment line as ‘delay’ (S10). The delay analyzing unit 137 may set the index to ‘delay’ for the average waiting time of the corresponding treatment line (S11).

As the result of the determination in the step (S6), if the average waiting time is less than or equal to the second reference value R2, the delay analyzing unit 137 determines that the treatment status of the corresponding treatment line is ‘normal’ (S12). The delay analyzing unit 137 may set the index to ‘normal’ for the average waiting time of the corresponding treatment line (S13).

The delay analyzing unit 137 determines whether the average treatment time corresponding to the medical shelter is greater than a fourth reference value R4 (S14).

If the result of the determination in the step (S14), if the average treatment time is greater than the fourth reference value R4, the delay analyzing unit 137 determines the treatment status of the corresponding treatment line as ‘delayed’ (S15). The delay analyzing unit 137 may set the index as ‘delay’ for the average treatment time of the treatment line (S16).

As the result of the determination in the step (S14), if the average treatment time is less than or equal to the fourth reference value R4, the delay analyzing unit 137 determines the treatment status of the corresponding treatment line as ‘normal’ (S17). The delay analyzing unit 137 may set the index to ‘normal’ for the average treatment time of the corresponding treatment line S18.

The delay analyzing unit 137 may determine the delay level of the corresponding treatment line by summing the indexes for each of the queue number, the average waiting time, and the average treatment time. The delay analyzing unit 137 converts each of the indices ‘normal’, ‘delay’, ‘busy’, and ‘severe’ into values (e.g., 0, 1, 2, 3), adds the converted values, and calculates an average thereof, thereby it is possible to determine the delay level as one of level 1 to level 4 according to the calculated average. For example, the delay level 1 may be set when the average is 0 or more and 0.5 or less, the delay level 2 may be set when the average is greater than 0.5 and 1.5 or less, the delay level 3 may be set when the average is greater than 1.5 and less than 2.5, and the delay level 4 may be set when the average is greater than 2.5 and 3 or less.

The patient analyzing unit 132 determines the patient status level by collecting the final examination result for the patient status received from the plurality of outlet devices 12_1 to 12_n and the treatment information from each medical shelter received from the plurality of monitoring devices 11_1 to 11_n. The patient analyzing unit 132 may receive the queue status information from the plurality of monitoring devices 11_1 to 11_n, derive the total treatment time that is the sum of the treatment times of the plurality of medical shelters constituting the treatment line in which the patient's treatment has been performed and the average treatment time that is the average thereof, and index the average treatment time and the total treatment time to patient status level.

The patient analyzing unit 132 may determine the patient status level as the patient status level 4 when the final examination result is in an emergency situation or an unstable condition of the elderly (70 years old or older) (1), as the patient status level 3 when there is an underlying disease and the patient is 50 or more and 70 years old or less (2), as the patient status level 2 when the patient is overweight (3), and as the patient status level 1 otherwise (4).

The line determining unit 133 determines the treatment line of the target vehicle 2 based on the delay level for each of the plurality of treatment lines received from the delay analyzing unit 137, the status level received from the patient status determining unit 135, and the indexed patient status level received from the patient analyzing unit 132.

The line determining unit 133 calculates a value (hereinafter, a weighted delay level) obtained by multiplying the delay level for each of the plurality of treatment lines by a weight value of the corresponding treatment line, and determines the treatment line having the smallest value among the weighted delay levels of the plurality of treatment lines as the treatment line of the target vehicle. The line determining unit 133 may set the weight value of each treatment line as the state level average of the patients in m (a natural number) vehicles which have recently entered each treatment line. Also, the line determining unit 133 may compensate the weight value in consideration of the patient status level, and the average treatment time and the total treatment time indexed to the patient status level. For example, the line determining unit 133 may compensate the weight value according to a ratio between reference values corresponding to each of the patient status level, the average treatment time, and the total treatment time. Specifically, when the patient status level is the patient status level 4 and the corresponding reference value is 2, the weight value may be increased by a % based on 2, which is the ratio therebetween. Here, “a” is a value proportional to the ratio between the patient status level and the reference value.

The line determining unit 133 may transmit the determined treatment line to the target vehicle 2.

In addition, the line determining unit 133 may store the information used for determining the line in the database 136.

The drive-through treatment system according to an embodiment may optimize the medical service provision time by guiding the treatment line by precisely checking the patient status and the condition of the treatment line. In addition, the drive-through treatment system may optimize the treatment required time and increase a full recovery treatment capability by storing and analyzing the data including the patient status and the treatment information.

While this disclosure has been described in connection with what is presently considered to be practical embodiments, it is to be understood that the disclosure is not limited to the disclosed embodiments. On the contrary, it is intended to cover various modifications and equivalent arrangements included within the spirit and scope of the appended claims.

What is claimed is:

1. A drive-through treatment system for a drive-through clinic, comprising:
a plurality of monitoring devices positioned at an inlet of a plurality of treatment lines;
a plurality of treatment devices positioned on a plurality of medical shelters in each of the plurality of treatment lines;
a plurality of outlet devices positioned at an outlet of the plurality of treatment lines; and
a treatment line determining device for determining a treatment line into which a target vehicle to enter the drive-through clinic will enter among the plurality of treatment lines based on information received from the plurality of monitoring devices and the plurality of outlet devices,
wherein each of the plurality of monitoring devices checks a treatment start, a treatment progress situation, and a treatment speed of vehicles that have entered the treatment line from the inlet of the corresponding treatment line, and transmits at least one of the treatment start, the treatment progress situation, or the treatment speed of the vehicles to the treatment line determining device, and
each of the plurality of outlet devices checks a final treatment time of the vehicles exiting the treatment line from the outlet of the corresponding treatment line, a remaining time moving from the inlet to the outlet, and a treatment result, and transmits at least one of the final treatment time, the remaining time, or the treatment result to the treatment line determining device.

2. The drive-through treatment system of claim 1, wherein:
each of the plurality of monitoring devices receives queue status information, a treatment required time for each of the vehicles in the queue, and treatment information of a doctor from each of the plurality of treatment devices of the corresponding treatment line, and synthesizes the received information to be transmitted to the treatment line determining device, and
the queue is a list of the vehicles that have entered the clinic where each of the plurality of treatment devices of the corresponding treatment line are positioned, the queue status information includes information on the number of the vehicles being treated in the corresponding medical shelter and a time of entering and exiting the queue of each of the vehicles, and the treatment required time for each of the vehicles in the queue is a treatment required time for each of the vehicles of the corresponding medical shelter.

3. The drive-through treatment system of claim 2, wherein each of the plurality of monitoring devices analyzes and checks a plurality of treatment information sets received from the plurality of treatment devices of the corresponding treatment line, and includes a data collection unit classifying and collecting the identified treatment information according to a predetermined collection format.

4. The drive-through treatment system of claim 3, wherein the data collection unit acquires information on the treatment status and the treatment required time of the medical shelter in which each of the plurality of treatment devices of the corresponding treatment line is positioned based on a plurality of queue situation information sets of the corresponding treatment line and the treatment required time for each of the vehicles in a plurality of queues.

5. The drive-through treatment system of claim 4, wherein the data collection unit collects the number of the vehicles receiving treatment from each medical shelter according to each of the plurality of queue situation information sets and the treatment required time for each of the vehicles in the plurality of queues to obtain a total treatment required time of each of the plurality of medical shelters of the corresponding treatment line.

6. The drive-through treatment system of claim 1, wherein each of the plurality of outlet devices includes:
an entry detection unit that checks the type and the number plate of the target vehicle entering the outlet of the corresponding treatment line, and the patient, and requests and receives the time-related information and the treatment information for the identified patient from the corresponding monitoring device; and
a parsing unit for separating the time-related information and the treatment information, and
the time-related information includes the information about the queue entry time point, the queue exiting time point, and a current queue value of the target vehicle in each of the plurality of medical shelters of the corresponding treatment line.

7. The drive-through treatment system of claim 6, wherein each of the plurality of outlet devices further includes:
a patient state analyzing unit that determines the state of the patient who has entered the outlet, and determines the final examination result in consideration of the identified patient state along with the treatment information received from the parsing unit; and
a queue delay analyzing unit that collects the time-related information to check the required time for each queue of the plurality of medical shelters of the corresponding treatment line and analyzes whether there is a delay in receiving the treatment for the target vehicle.

8. The drive-through treatment system of claim 7, wherein the queue delay analyzing unit determines that there is a delay in the treatment at the medical shelter corresponding to the corresponding queue if the time from the time point when the target vehicle enters the queue corresponding to each of the plurality of medical shelters of the corresponding treatment line to the time point when the target vehicle comes out of the corresponding queue is greater than or equal to a predetermined threshold value.

9. The drive-through treatment system of claim 7, wherein the queue delay analyzing unit determines that there is a delay in moving between two adjacent medical shelters corresponding to the previous queue and the corresponding queue when the time difference between the time when the target vehicle exits from the previous queue and the time when the target vehicle enters the corresponding queue is greater than or equal to a predetermined threshold value based on the queue corresponding to each of the plurality of medical shelters of the corresponding treatment line.

10. The drive-through treatment system of claim 1, wherein the treatment line determining device includes:
a data reallocation unit performing data processing and data formatting on the data received from the target vehicle;
a patient status determining unit for receiving the patient status of the target vehicle and position information on the movement path of the target vehicle from the data reallocation unit, and determining a status level indicating the patient's status level of the target vehicle; and
a queue analyzing unit for analyzing the current delay situation in the plurality of medical shelters in each of the plurality of treatment lines, and
the queue is a list of the vehicles that have entered the clinic where each of the plurality of treatment devices of each of the plurality of treatment lines is positioned.

11. The drive-through treatment system of claim 10, wherein the queue analyzing unit receives each queue status information set of the entire medical shelter that is the plurality of medical shelters of an entirety of the plurality of treatment lines from the plurality of monitoring devices, and derives the number of each queue of the entire medical shelter, the waiting time, and the treatment time.

12. The drive-through treatment system of claim 11, wherein the queue analyzing unit, in two adjacent medical shelters of the plurality of medical shelters in one treatment line, derives a period between the time point when the target vehicle exits from the previous queue, which is the queue of the medical shelter positioned first in the target vehicle progress direction and the time point when the target vehicle enters the next queue, which is the queue of the medical shelter positioned next in the target vehicle progress direction, as the waiting time of the next medical shelter.

13. The drive-through treatment system of claim 11, wherein the queue analyzing unit derives the period between the time point when the target vehicle enters the queue of the medical shelter and the time point when it leaves the same queue as the treatment time of the medical shelter.

14. The drive-through treatment system of claim 11, wherein the treatment line determining device further includes a delay analyzing unit for determining a delay level for each of the plurality of treatment lines based on the number of the queues for each of the entire medical shelters, the waiting time, and the treatment time, respectively.

15. The drive-through treatment system of claim 14, wherein the delay analyzing unit derives the number of the queues by adding up each number of the queues of the entire medical shelters that is the plurality of medical shelters in an entirety of the plurality of treatment lines, and derive an average of the waiting time and the treatment time of the plurality of medical shelters constituting each of the plurality of treatment lines.

16. The drive-through treatment system of claim 15, wherein the delay analyzing unit compares each of the queue number, the average waiting time, and the average treatment time of each of the plurality of treatment lines with a corresponding reference value to determine a delay level indicating a delay degree of each of the plurality of treatment lines based on each of the queue number, the average waiting time, and the average treatment time.

17. The drive-through treatment system of claim 16, wherein the delay analyzing unit determines that the treatment status of the treatment line is busy when the number of the queues in one of the plurality of treatment lines is greater than the first reference value, and the treatment status of the treatment line is normal when it is less than the first reference value.

18. The drive-through treatment system of claim 14, wherein the treatment line determining device further includes a patient analyzing unit determining the patient status level by collecting the final examination result for the patient status received from the plurality of outlet devices and the treatment information received from the plurality of monitoring devices, derives the total treatment time that is the sum of the treatment times of the plurality of medical shelters constituting the treatment line in which the treatment of the patient has been performed and the average treatment time that is the average thereof, and indexes the average treatment time and the total treatment time to the patient status level.

19. The drive-through treatment system of claim 14, wherein the treatment line determining device further includes a line determining unit to determine the treatment line of the target vehicle among the plurality of treatment lines based on the delay level of each of the plurality of treatment lines received from the delay analyzing unit and the status level received from the patient status determining unit.

20. The drive-through treatment system of claim 14, wherein:
the treatment line determining device further includes a patient analyzing unit determining the patient status level by collecting the final examination result for the patient status received from the plurality of outlet devices and the treatment information received from the plurality of monitoring devices, derives the total treatment time that is the sum of the treatment times of the plurality of medical shelters constituting the treatment line in which the treatment of the patient has been performed and the average treatment time that is the average thereof, and indexes the average treatment time and the total treatment time to the patient status level; and
a line determining unit for determining the treatment line of the target vehicle among the plurality of treatment lines based on the delay level of each of the plurality of treatment lines received from the delay analyzing unit, the status level received from the patient status determining unit, and the indexed patient status level received from the patient analyzing unit.
